SEQUENCE LISTING (1 sequence)

SEQ ID NO: 1 - DNA, 147 bp, Artificial Sequence
  mutant Polyoma virus enhancer
ctcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgag

(19) United States
(12) Patent Application Publication
(10) Pub. No.: US 2003/0158139 A1
(43) Pub. Date: Aug. 21, 2003

(54) Decreasing adipose mass by altering RSK2 activity

(76) Inventors: Christian Bjorbaek, Boston, MA (US); Laurie J. Goodyear, Brewster, MA (US); Jeffrey S. Flier, West Newton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/261,092

(22) Filed: Sep. 27, 2002

Related U.S. Application Data
(63) Continuation of application No. 10/016,692, filed on Oct. 30, 2001, now abandoned.
Application No. 10/016,692 is a continuation of application No. PCT/US00/11679, filed on May 1, 2000.
(60) Provisional application No. 60/131,762, filed on Apr. 30, 1999.

(51) Int. Cl. A61K 48/00; C12Q 1/68; G01N 33/53

(52) U.S. Cl. 514/44; 435/6; 435/7.1

(57) ABSTRACT

Deletion of the rsk2 gene in mice results in reduced body weight, reduced body fat and reduced sensitivity to diet-induced weight gain, as well as lower levels of leptin in the serum of rsk2 deficient mice and lower levels of oxygen consumption, as compared to wild type littermates. Thus, altering RSK2 activity provides a means for modulating RSK2-mediated signaling and therefore modulating the above described physiological parameters. The present invention encompasses methods and compositions for altering, or modulating in a mammal, body weight, fat content, leptin levels by altering, or modulating, RSK2 activity. Specifically encompassed in the present invention are methods and compositions to alter activity of the RSK2. The present invention is drawn to a model for the study of Coffin-Lowry syndrome as well as an in vivo model to screen and test therapeutic agents for the treatment of Coffin-Lowry syndrome. The present invention is further drawn to use of the rsk2 knockout mouse as a model to study and treatment of lipodystrophy and impaired glucose tolerance in mammals.

### RELATED APPLICATIONS

This application is a continuation of U.S. application Ser. No. 10/016,692, filed Oct. 30, 2001, which is a continuation of International Application No. PCT/US00/11679, which designated the United States and was filed on May 1, 2000, which claims the benefit of U.S. Provisional Application No: 60/131,762, filed Apr. 30, 1999. The entire teachings of the above applications are incorporated by reference.

### GOVERNMENT SUPPORT

This invention was supported, in whole or in part, by a grant RO1 45874-02, AR42238 and DK28082 all from the National Institutes of Health. The Government has certain rights in the invention.

### BACKGROUND OF THE INVENTION

Ribosomal-S6-kinases (p90^{rsk} or RSK) are intracellular serine/threonine protein kinases that are activated by perturbations including stimulation with insulin and other growth factors. Three members of the RSK family have been identified (RSK1, RSK2, RSK3). RSK proteins are expressed in many tissues. RSK2 is highly expressed in brain, muscle and fat tissue. RSK2 is activated, for example, in response to the growth factors via the MAPK pathway and possibly other pathways. Studies in mammalian cell lines show that RSK isoforms, upon activation, translocate to the nucleus where they may be involved in phosphorylation of transcription factors. Putative RSK substrates include histones as well as transcription factors like c-Fos, c-Jun, Elk-1 and serum response factor. RSK2 acts as a CREB (cAMP-responsive binding-element protein) kinase in response to the growth factors via the MAPK pathway. These results implicate a role of RSK proteins as mediators of cellular proliferation.

Mutations in the rsk2 gene have recently been demonstrated to cause Coffin-Lowry syndrome, an X-linked disorder characterized by mental retardation, short stature and digital dysmorphisms. Yet the function of RSK2 in vivo remains largely unknown.

### SUMMARY OF THE INVENTION

The present invention encompasses methods and compositions for altering, or modulating in a mammal, body weight, fat content, leptin levels or oxygen consumption by altering, or modulating, RSK2 activity. Specifically encompassed in the present invention are methods and compositions to alter activity of the RSK2. As demonstrated herein, it has now been determined that deletion of the rsk2 gene in mice results in reduced body weight, reduced body fat and reduced sensitivity to diet-induced weight gain, as well as lower levels of leptin in the serum of rsk2 deficient mice, as compared to wild type littermates. Thus, as described herein, altering RSK2 activity provides a means for modulating RSK2-mediated signaling and therefore modulating the above described physiological parameters.

The present invention is further drawn to a model for the study of Coffin-Lowry syndrome as well as an in vivo model to screen and test therapeutic agents for the treatment of Coffin-Lowry syndrome.

The rsk2 knockout mice have a diabetic-like phenotype. In addition, the rsk2 knockout mice have characteristics of lipodystrophy, a condition which can lead to diabetes in humans. Therefore, the present invention is also drawn to a model for lipodystrophy and diabetes related to lipodystrophy and a method to test compound in vivo for their ability to affect symptoms of lipodystrophy. The method of testing compounds in vivo for their ability to affect symptoms of lipodystrophy comprises administering said compound to a rsk2 knockout mouse and measuring the effect on phenotypic parameters of lipodystrophy in the treated mice. Compounds that result in altered physiological parameters associated with lipodystrophy are then selected.

The present invention is drawn to use of a RSK2 inhibitor for the manufacture of a medicament for the treatment of obesity; reduction of body weight; reduction body fat; reduction of serum leptin levels in a mammal. In one embodiment, the RSK2 inhibitor comprises a polynucleotide construct comprising a polynucleotide that prevents transcription of rsk2 DNA; or a polynucleotide encoding a rsk2 antisense polynucleotide; or a polynucleotide encoding a modified RSK2 polypeptide, wherein said modified RSK2 polypeptide is a competitive inhibitor of endogenous RSK2 activity; or a RSK2 inhibitor, wherein the inhibitor interferes with the interaction of RSK2 with a RSK2 target protein.

The present invention is drawn to a method of identifying inhibitors of RSK2 activity. In the method of the present invention, cells having at least some RSK2 activity are contacted with an organic molecule library comprising candidate RSK2 inhibitors or transfecting said cells with a cDNA expression library comprising DNA encoding candidate RSK2 inhibitors. Cells having decreased RSK2 activity are selected and the organic molecule or DNA selected with the cells having decreases RSK2 activity is identified.

The present invention is drawn to a method of testing compounds in vivo for their ability to inhibit RSK2 activity. The method comprises the steps of administering at least one candidate compound to a mouse having at least some level of RSK2 activity and measuring RSK2 activity in the treated mice. Compounds that result in altered RSK2 activity are selected.

The present invention is drawn to RSK2 inhibitors and compounds identified by the methods of the present invention.

RSK activity can be inhibited by inhibiting or reducing the amount of RSK protein expressed in a cell or cells or tissues of an organism. RSK activity can further be inhibited by introducing a polynucleotide encoding a modified RSK protein into a cell, wherein the modified RSK2 protein comprises a mutant, variant, derivative, or analog of the RSK2 protein that is a competitive inhibitor of RSK2. The competitive inhibitor may interfere with upstream activating elements of RSK2, such as proteins in the MEK-MAPK pathway, or downstream targets of RSK2, such as protein phosphatase 1 (involved in insulin-stimulated activation of glycogen synthase) as well as c-Jun, c-Fos and serum response factor (involved in growth factor regulation of cell proliferation).

RSK2 expression can be inhibited or reduced by transfecting a cell with a polynucleotide construct encoding rsk2 antisense DNA or RNA. For example, the antisense RNA can hybridize to the endogenous rsk2 mRNA and prevent translation of rsk2 mRNA, thereby inhibiting or reducing expression of RSK2 protein. RSK2 expression can also be inhibited or reduced by transfecting the cell with a polynucleotide construct encoding a transcriptional inhibitor such that transcription of RSK2 is inhibited or reduced. Such a transcriptional inhibitor would interact specifically with rsk2 promoter sequences, resulting in decreased transcription of rsk2, decreased RSK2 protein expression and thus decreased RSK2 activity. Methods of making and administering antisense molecules and transcriptional inhibitors are well known in the art. Rsk2 transcription can also be inhibited by administering peptide nucleic acid (PNA) that specifically hybridizes to and inhibits the transcription of rsk2. Methods of making specific PNA are well known in the art.

RSK2 activity can also be inhibited by transfecting the cell with a polynucleotide construct encoding an altered, or modified RSK2 protein, polypeptide or peptide. In one embodiment, the modified RSK2 polypeptide is a competitive inhibitor (e.g., antagonist) of endogenous RSK2. The modified RSK2 can interact with a RSK2 target protein, without interfering with the activity of the target protein. Because the modified RSK2 protein interacts with the intended RSK2 target, endogenous RSK2 could not interact with its intended target, thereby inhibiting or reducing the level of RSK2 mediated cell signaling. In another embodiment, RSK2 activity can be inhibited or reduced by introducing a RSK2 inhibitor into the cell. Such an inhibitor can be a peptide or small organic molecule that interferes with RSK2 activity. Such an inhibitor can interact specifically with RSK2, or to its intended target, to inhibit RSK2 activity. For example, the inhibitor can interact with downstream targets of RSK2 such as protein phosphatase 1, Elk-1, c-Jun or c-Fos. In still another embodiment, the antagonist or inhibitor can interact with upstream activators of RSK2, such as MAPK thereby preventing the activation of RSK2.

The present invention further encompasses methods of increasing or enhancing RSK2 activity in a cell or the cells or tissues of an organism. Increased RSK2 activity in a cell is reasonably expected to increase body weight, fat content and leptin levels in a mammal, and increase insulin sensitivity. RSK2 activity can be increased by transfecting a cell or cells or tissues of an organism with a polynucleotide construct encoding a biologically active form of RSK2 protein, or a biologically active fragment thereof. In another embodiment, RSK2 activity can be increased by transfecting a cell with a nucleic acid encoding a modified RSK2 protein that has increased biological activity.

The present invention also pertains to an inbred mouse strain wherein the rsk2 gene has been disrupted by homologous recombination. This rsk2 “knockout” (KO) mouse can be used to evaluate RSK2 activity and to screen candidate RSK2 inhibitors, antagonists and agonists for activity by comparison with the activities of these molecules on mice that are identical except for the presence of a wild type copy of the rsk2 gene. These rsk2 knockout mice are further useful for the study of Coffin-Lowry syndrome, lipodystrophy and lipodystrophy related diabetes as described above. Furthermore, the rsk2 knockout mice of the present invention are useful to produce cell lines that can be used to study the RSK2 enhancers and inhibitors of the present invention.

Cell lines, from rsk2 knockout mice (see Bruning et al., (2000), Proc Natl Acad Sci USA. 97:2462-7) and genetically identical mice that are wild type for the rsk2 gene can be used to screen libraries such as organic molecule libraries or cDNA libraries to select and identify molecules that inhibit (or enhance) RSK2 activity.

Thus, as a result of the discovery described herein, methods and compositions are now available to modulate or compensate RSK2 activity, specifically by modulating the activity of RSK2, or by modulating downstream targets of RSK2.

### BRIEF DESCRIPTION OF THE DRAWINGS

FIG. 1A is a schematic drawing of the genomic structure of rsk2, the targeting vector and the targeted structure of the disrupted rsk2 gene.

FIG. 1B is a schematic diagram of the diagnostic fragments from wild type and recombinant (disrupted) rsk2 genes.

FIG. 1C is a Southern blot of Hind III-digested genomic DNA from wild type and knockout (KO) male and female mice.

FIG. 1D is a schematic diagram of the RSK2 protein, where the gene knock out results in neo insertion in the kinase domain 1, resulting in truncated RSK2 protein.

FIG. 1E shows a Western blot for RSK2 protein expression in muscle tissue from wild type male (+/Y) and wild type female (+/+), KO male (−/Y) and KO female (−/−) mice.

FIG. 2 is a schematic drawing of the plasmid used to make the targeting vector.

FIG. 3 is a Western blot of RSK2 protein from muscle and white adipose tissue (WFT) from wild type and KO mice, as well as from brain-derived cell lines comprising astrocytes and GT-7 cells.

FIG. 4A is a bar graph showing the results of a coordination test administered to 20 week old wild type and KO mice, where the filled bars represent wild type mice.

FIG. 4B is a bar graph showing results of a cognitive function test, administered to 10 week old wild type (filled bars) and KO mice.

FIG. 5A shows weight gain over time in wild type (filled boxes) and KO (open circles) mice, *p<0.01.

FIG. 5B shows nose to anus length in 8 week old male wild type and KO mice, *p<0.01.

FIG. 6A shows rectal body temperatures of wild type and KO mice.

FIG. 6B shows oxygen consumption by individual, live wild type and KO mice, *p<0.03.

FIG. 6C shows UCP1, UCP2 and UCP3 mRNA expression levels in brown fat (BFT), white fat (WFT) and muscle tissue (Musc.) from wild type (filled bars) and KO mice.

FIG. 7A shows the level of food intake by wild type (filled bars) and KO mice at age 6 and 20 weeks.

FIG. 7B shows total hypothalamic mRNA expression of neuropeptides: neuropeptide Y (NPY), agouti-related peptide (AGRP) and proopiomelanocortin (POMC), as measured by quantitative RT-PCR on total mRNA isolated from hypothalami from wild type (filled bars) and KO mice.

FIG. 7C shows the level of circulating serum leptin levels in 6 week old wild type (filled bars) and KO mice, *p<0.01.

FIG. 8A shows epididymal fat pad weights of wild type (filled bars) and KO mice at 8, 25 and 36 weeks of age, *p<0.01.

FIG. 8B shows fat cell numbers in wild type (filled bars) and KO mice at 8, 25 and 36 weeks of age, *<0.04.

FIG. 8C shows fat cell size in wild type (filled bars) and KO mice at 8, 25 and 36 weeks of age, *p<0.01.

FIG. 9 shows a Western blot for activated Capase-3 in white adipose tissue (WAT) muscle tissue or brain tissue from wild type and KO mice.

FIG. 10A shows the results of a glucose tolerance test (GTT) on 18 week old wild type (filled boxes) and KO (open boxes), p<0.01.

FIG. 10B shows fasting glucose levels in 18 week old wild type (filled bar) and KO mice, p<0.05.

FIG. 10C shows fasting insulin levels in 18 week old wild type (filled bar) and KO mice, p<0.01.

FIG. 11A shows body weight of wild type (filled squares) and KO (open diamonds) mice, after the indicated days on a high fat diet, *p<0.01.

FIG. 11B shows food intake by wild type (filled squares) and KO (open circles) mice after the indicated days on a high fat diet.

FIG. 11C shows total body fat measured in wild type (filled bar) and KO mice after 70 days on a high fat diet, p<0.05.

FIG. 11D shows leptin levels in wild type (filled bar) and KO mice after 70 days on a high fat diet, p<0.04.

FIG. 12A shows MAPK (ERK) phosphorylation in muscle in wild type and KO mice 10 minutes and 30 minutes after insulin injection.

FIG. 12B shows densitometric quantification of the ERK1 bands of FIG. 11A, *p<0.001, #p<0.05.

FIG. 12C shows densitometric quantification of the ERK2 bands of FIG. 11B, *p<0.001, #p<0.05.

FIG. 13A is a Western blot showing Akt Ser phosphorylation in wild type and KO mouse muscle 10 minutes after insulin injection.

FIG. 13B shows densitometric quantification of FIG. 12A, *p<0.02 using unpaired T-test.

FIG. 14A shows insulin receptor phosphorylation in wild type and KO mouse muscle before and 10 minutes after insulin treatment.

FIG. 14B shows IRS1 phosphorylation in wild type and KO mouse muscle before and 10 minutes after insulin treatment.

FIG. 14C shows IRS/p85 association in wild type and KO mouse muscle before and 10 minutes after insulin treatment.

### DETAILED DESCRIPTION OF THE INVENTION

Stimulation by growth factors of the Ras-MAPK signaling pathway results in activation of p90 Ribosomal S6 Kinases (RSK's). At least 4 members of the protein family are known, with RSK2 being highly expressed in brain, fat and muscle tissue. Studies suggest that RSK2 promotes cell survival in neuronal and non-neuronal cells. Recently, mutations in the rsk2 gene have been associated with the Coffin-Lowry syndrome (CLS), a form of X-linked mental retardation. To further study the function of RSK2 in vivo, we generated rsk2 knockout (KO) mice. rsk2 KO animals have a reduced learning capacity as measured by standard tests. Birth-weights of rsk2 KO mice are normal, but the growth rate after weaning is reduced. KO animals have increased oxygen consumption, without changes in food intake, body temperature and mRNA expression of uncoupling proteins (UCP1-3). When compared to wild type (WT) littermates, rsk2 KO mice have significantly reduced amounts of white fat tissue (more than 50%), with decreases in both adipocyte cell size and number. These changes become more pronounced with age and are accompanied by reduced serum leptin levels. Western blotting analysis demonstrate that rsk2 KO mice have increased amounts of activated Capase-3 protein levels in adipose, but not in brain or muscle tissue. Furthermore, KO mice have impaired glucose tolerance and elevated fasting insulin levels (2 fold). Finally, when exposed to a high-fat-diet, rsk2 KO mice demonstrate resistance to weight gain, largely due to less accumulation of fat mass. Mice lacking functional RSK2 have a reduced learning capacity consistent with human CLS. In addition, rsk2 knockout mice have reduced fat mass and insulin resistance. The increase in activated Capase-3 in fat tissue suggest that RSK2 promotes survival of adipocytes in vivo. The reduction in fat tissue may negatively affect insulin sensitivity, as observed in lipodystrophy, possibly through reduced levels of adipocyte-derived factors such as leptin. RSK2 also appears to play a role in negative feedback regulation of the Akt and MAPK pathways in muscle tissue, without affecting proximal insulin signaling.

The present invention encompasses methods and compositions for modulating body weight and fat content comprising altering RSK2 activity. As defined herein, modulating (also referred to herein as altering, adjusting or regulating) RSK2 activity means inhibiting or enhancing the biological activity of RSK2. Inhibiting RSK2 activity encompasses partial inhibition as well as complete abrogation of RSK2 activity. Enhancing RSK2 activity encompasses measurable increases in RSK2 activity. These increases can be measured in a range of background activities from complete absence of RSK2 activity to wild type levels of RSK2 activity. The inhibition or increase in RSK2 activity can last from a few hours to many years in the organism or cells of interest.

The biological activity of RSK2 is defined herein as the ability of RSK2 to activate one or more signal transduction pathways in a cell, or cells or tissues of an organism. The signal transduction pathways are, for example, insulin-stimulated activation of glycogen synthase, growth factor regulation of cell proliferation, cell survival and growth factor stimulated autophosphorylation, and phosphorylation of Akt and MAPK. Biological activity also includes the ability of RSK2, or fragments thereof, to be bound by antibodies (antigenic) or to stimulate an immune response (immunogenic), wherein the immune response includes the production of RSK2 specific antibodies. RSK2 activity can further be determined by comparing the level of leptin or insulin in the serum of the organism before and after treatment with the RSK2 inhibitor or activator. Furthermore, RSK2 activity can be determined by measuring the fat content of the organism before and after treatment with the RSK2 inhibitor or activator.

As defined herein, modified RSK2 encompasses RSK2 molecules comprising fragments, derivatives, analogs, variants and mutants of the RSK2 protein. These modified RSK2 molecules possess RSK2 inhibitor/antagonist activity, thereby inhibiting the activity of endogenous RSK2 present in a cell, resulting in above described physiological changes. Another activity of modified RSK2 molecules can be the antigenic property of the modified RSK2 molecule comprising the ability of the modified RSK2 to bind to RSK2-specific antibodies. The modified RSK2 molecule can also possess immunogenic properties whereby the modified RSK2 molecule induces an immunogenic response, e.g., the production of antibodies that specifically bind to endogenous (native) RSK2.

A fragment of RSK2 encompasses polypeptides that comprise only a part of the full-length RSK2 protein and inhibits endogenous RSK2 activity. Such fragments can be produced by amino and/or carboxyl terminal deletions, as well as internal deletions. Fragments can also be produced by enzymatic digestion. Such modified RSK2 molecules can be tested for inhibitory activity as described herein.

“Derivatives” and “variants” of RSK2 can include truncated and hybrid forms of RSK2. “Truncated” forms are shortened forms of RSK2, typically with deletions of regions of the protein as described above. “Hybrid” forms of RSK2 are RSK2 molecules comprising a portion of a RSK2 amino acid sequence with non-RSK2 amino acid sequence, e.g., RSK1, RSK3, or non RSK sequence.

“Variants” and “mutants” of RSK2 can be produced using in vitro and in vivo techniques well-known to those of skill in the art, for example, site-specific mutagenesis and oligonucleotide mutagenesis. Manipulations of the RSK2 protein sequence can be made at the protein level as well. Any numerous chemical modifications can be carried out by known techniques including, but not limited to, specific chemical cleavage by cyanogen bromide, trypsin and papain. RSK2 can also be structurally modified or denatured, for example, by heat. In general, mutations can be conservative or non-conservative amino acid substitutions, amino acid insertions or amino acid deletions. The mutations can be at or near RSK2 binding sites.

For example, DNA encoding a Rsk2 mutant is prepared by site-directed mutagenesis of DNA that encodes RSK2. Site-directed (site-specific) mutagenesis allows the production of RSK2 variants through the use of specific oligonucleotide sequences that encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 20 to 25 nucleotides in length is preferred, with about 5 to 10 residues on both sides of the junction of the sequence being altered. In general, the techniques of site-specific mutagenesis are well known in the art, as exemplified by publications such as Edelman et al., DNA 2:183, 1983. The site-specific mutagenesis technique typically employs a phage vector that exists in both a single-stranded and double-stranded form. Typical vectors useful in site-directed mutagenesis include vectors such as the M13 phage, for example, as disclosed by Messing et al., Third Cleveland Symposium on Macromolecules and Recombinant DNA, A. Walton, ed., Elsevier, Amsterdam, 1981. This and other phage vectors are commercially available and their use is well-known to those skilled in the art. A versatile and efficient procedure for the construction of oligonucleotide directed site-specific mutations in DNA fragments using M13-derived vectors was published by Zoller, M. J. and Smith, M., Nucleic Acids Res. 10:6487-6500, 1982. Also, plasmid vectors that contain a single-stranded phage origin of replication can be employed to obtain single-stranded DNA, Veira et al., Meth Enzymol. 153:3 1987.

In general, site-specific mutagenesis herewith can be performed by first obtaining a single-stranded vector that includes within its sequence a DNA sequence that encodes the relevant protein. An oligonucleotide primer bearing the desired mutated sequence is prepared, generally synthetically, for example, by the method of Crea et al., Proc Natl Acad Sci USA. 75:5765, 1978. This primer can then be annealed with the single-stranded protein sequence-containing vector, and subjected to DNA polymerizing enzymes such as E. coli polymerase I Klenow fragment, to complete the synthesis of the mutation-bearing strand. Thus, a heteroduplex is formed wherein one strand encodes the original non-mutated sequence and the second strand bears the desired mutation. This heteroduplex vector can then be used to transform appropriate host cells such as JM 101 cells, and clones can be selected that include recombinant vectors bearing the mutated sequence arrangement. Thereafter, the mutated region can be removed and placed in an appropriate expression vector for protein production.

The PCR technique can also be used in creating amino acid sequence variants of RSK2. When small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template. For introduction of a mutation into a plasmid DNA, one of the primers can be designed to overlap the position of the mutation and to contain the mutation; the sequence of the other primer is preferably identical to a stretch of sequence of the opposite strand of the plasmid, but this sequence can be located anywhere along the plasmid DNA. It is preferred, however, that the sequence of the second primer is located within 500 nucleotides from that of the first, such that in the end the entire amplified region of DNA bounded by the primers can be easily sequenced. PCR amplification using a primer pair like the one just described results in a population of DNA fragments that differ at the end position of the mutation specified by the primer.

The DNA fragments produced bearing the desired mutation can be used to replace the corresponding region in the plasmid that served as PCR template using standard DNA technology. Mutations at separate positions can be introduced simultaneously by either using a mutant second primer or performing a second PCR with different mutant primers and ligating the two resulting PCR fragments simultaneously to the vector fragment in a three (or more) part ligation.

Another method for preparing variants, cassette mutagenesis, is based on the technique described by Wells et al. Gene 34, 315, 1985. The starting material can be the plasmid (or vector) comprising the rsk2 DNA to be mutated. The codon(s) within the rsk2 to be mutated are identified. There must be unique restriction endonuclease sites on each side of the identified mutation site(s). If such restriction sites do not exist, they can be generated using the above-described oligonucleotide-mediated mutagenesis method to introduce them at appropriate locations in the rsk2 DNA. After the restriction sites have been introduced into the plasmid, the plasmid is cut at these sites to linearize it. A double stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are compatible with the ends of the linearized plasmid, such that it can be directly ligated to the plasmid. The plasmid now contains the mutated rsk2 DNA sequence, that can be expressed to produce RSK2 with altered binding activity.

The inhibitor compounds of the present invention include any molecule that interacts with endogenous RSK2 or to RSK2 target molecules described herein, such that upon interacting with said molecules, inhibitors of RSK2 mediate inhibition of RSK2 kinase activity and cell-signaling activity. Encompassed by the present invention are inhibitor compounds that mimic the structure and conformation of the substrate moiety when interacting with the RSK2 binding or active site. Molecular inhibitors of the present invention will typically have an inhibition constant (Kᵢ) of ten micromolar, or less. Specifically encompassed are organic molecules that mimic the structure and conformation of binding domains or substrate conformation and interact with RSK2, thereby inhibiting its activity. In one embodiment the inhibitor contains or mimics phosphothreonine or phosphoserine.

Also encompassed by the present invention are small organic molecules that mimic the structure of RSK2, or, alternatively, the binding site of the RSK2 target, and therefore, interfere with the interaction of RSK2 with its intended target molecule.

Peptides suitable for use as RSK2 inhibitors can be produced in libraries. The libraries of peptides comprise a mixture of substantially equimolar amounts of peptides. In one embodiment, the library can be designed or selected for molecules that mimic RSK2 target molecules. In another embodiment, the library comprises or is selected for peptides that interact with RSK2, thereby inhibiting the ability of RSK2 to bind target molecules.

The inhibitors of the present invention can be synthesized using standard laboratory methods that are well known to those of skill in the art, including standard solid phase techniques. Inhibitors comprising naturally occurring amino acids can also be produced by recombinant DNA techniques known to those of skill, and subsequently phosphorylated.

The inhibitors of the present invention can comprise either the 20 naturally occurring amino acids or other synthetic amino acids. Synthetic amino acids encompassed by the present invention include, for example, naphthylalanine, L-hydroxypropylglycine, L-3,4-dihydroxyphenylalanyl, α-amino acids such as L-α-hydroxylysyl and D-α-methylalanyl, L-α-methyl-alanyl, β amino-acids such as β-analine, and isoquinolyl.

D-amino acids and other non-naturally occurring synthetic amino acids can also be incorporated into the inhibitors of the present invention. Such other non-naturally occurring synthetic amino acids include those where the naturally occurring side chains of the 20 genetically encoded amino acids (or any L or D amino acid) are replaced with other side chains of the 20 genetically encoded amino acids (or any L or D amino acid) are replaced with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic. In particular, proline analogs in which the ring size of the proline residue is changed from 5 members to 4, 6, or 7 member can be employed.

As used herein, “lower alkyl” refers to straight and branched chain alkyl groups having from 1 to 6 carbon atoms, such as methyl, ethyl propyl, butyl and so on. “Lower alkoxy” encompasses straight and branched chain alkoxy groups having from 1 to 6 carbon atoms, such as methoxy, ethoxy and so on.

Cyclic groups can be saturated or unsaturated, and if unsaturated, can be aromatic or non-aromatic. Heterocyclic groups typically contain one or more nitrogen, oxygen, and/or sulphur heteroatoms, e.g., furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. The heterocyclic groups can be substituted or unsubstituted. Where a group is substituted, the substituent can be alkyl, alkoxy, halogen, oxygen, or substituted or unsubstituted phenyl. (See U.S. Pat. No. 5,654,276 and U.S. Pat. No. 5,643,873, the teachings of which are herein incorporated by reference).

Peptide mimetics that mimic the RSK2 protein can also be designed to inhibit RSK2 activity. These mimetics can be designed and produced by techniques known to those of skill in the art. (See e.g., U.S. Pat. Nos. 4,612,132; 5,643,873 and 5,654,276, the teachings of which are herein incorporated by reference). These mimetics are based on the RSK2 sequence, and possess activity antagonistic to the biological activity of the corresponding peptide compound, but possess a “biological advantage” over the corresponding peptide inhibitor with respect to one, or more, of the following properties: solubility, stability, and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic inhibitor. Examples of modifications of peptides to produce peptide mimetics are described in U.S. Pat. Nos. 5,643,873 and 5,654,276, the teachings of which are incorporated herein by reference. Peptide mimetic libraries can also be produced as described above.

Alternatively, the RSK2 inhibitor can be an antibody or antibody fragment that interacts with RSK2, thereby preventing RSK2 from interacting with downstream target molecules. The term “antibody” is meant to encompass polyclonal antibodies, monoclonal antibodies (mAbs), chimeric antibodies (e.g., humanized antibodies) and antibody fragments that retain the biological activity of specific binding to RSK2, such as Fab, Fab′, F(ab′)2 and Fv. Also encompassed are single-chain antibodies (sFvs). These antibody fragments lack the Fc portion of an intact antibody, clear more rapidly from the circulation and can have less non-specific tissue binding than an intact antibody. These fragments are produced by well-known methods in the art, for example by proteolytic cleavage with enzymes such as papain (to produce Fab fragments) or pepsin (to produce F(ab′)2 fragments.

Polyclonal antibodies are heterogeneous populations of antibody molecules derived from the sera of animals immunized with an antigen. A monoclonal antibody (mAb) contains a substantially homogenous population of antibodies specific to antigens, which population contains substantially similar epitope binding sites. MAbs may be obtained by methods known to those skilled in the art. See, for example Kohler and Milstein, Nature 256:495-497, 1975; U.S. Pat. No. 4,376,110; Ausubel et al, eds., Current Protocols in Molecular Biology, Green Publishing Assoc. and Wiley Interscience, N.Y., 1987, 1992; and Harlow and Lane Antibodies: A Laboratory Manual Cold Spring Harbor Laboratory, 1988; Colligan et al., eds., Current Protocols in Immunology, Greene Publishing Assoc. and Wiley Interscience, N.Y., 1992, 1993; the contents of which references are incorporated entirely herein by reference. Such antibodies can be of any immunoglobulin class including IgG, IgM, IgE, IgA, and any subclass thereof. A hybridoma producing a mAb of the present invention can be cultivated in vitro, in situ, or in vivo. Production of high titers of mAbs in vivo or in situ makes this the presently preferred method of production.

Chimeric antibodies which include humanized antibodies, are molecules wherein different portions of which are derived from different animal species, such as those having variable regions derived from a murine mAb and a human immunoglobulin constant region. Chimeric antibodies are primarily used to reduce immunogenicity in application and/or to increase yields in production, for example. Chimeric antibodies and methods for their production are known in the art (Cabilly et al., Proc Natl Acad Sci USA 81:3273-3277, 1984; Morrison et al., Proc Natl Acad Sci USA 81:6851-6855, 1984; Boulianne et al., Nature 312:643-646, 1984; Cabilly et al., European Patent Application 125023 (published Nov. 14, 1984); Neuberger et al., Nature 314:268-270, 1985; Taniguchi et al., European Patent Application 171496 (published Feb. 19, 1985); Morrison et al., European Patent Application 1739494 (published Mar. 5, 1986); Neuberger et al., PCT Application WO 86/01533, (published Mar. 13, 1986); Kudos et al., European Patent Application 184187 (published Jun. 11, 1986); Sahagan et al., J Immunol. 137:1066-1074, 1986; Robinson et al., International Patent Publication #PCT/US86/02269 (published May 7, 1987); Liu et al., Proc Natl Acad Sci USA 84:3439-3443, 1987; Sun et al., Proc Natl Acad Sci USA 84:214-218, 1987; Better et al., Science 240:1041-1043, 1988; and Harlow and Lane Antibodies: A Laboratory Manual, Cold Spring Harbor Laboratory, 1988. These references are entirely incorporated herein by reference.

Typically, antibodies of the present invention are high affinity anti-RSK2 antibodies, and fragments or regions thereof, that have potent inhibiting and/or neutralizing activity in vivo against RSK2. Such antibodies can include those generated by immunization using purified recombinant RSK2 or peptide fragments thereof.

Methods for determining antibody specificity and affinity can be found in Harlow, et al., Antibodies: A Laboratory Manual, Cold spring Harbor Laboratory Press, Cold Spring Harbor, N.Y., 1988; Colligan et al., eds., Current Protocols in Immunology, Greene Publishing Assoc. and Wiley Interscience, N.Y., 1992, 1993; and Muller, Meth. Enzymol., 92:589-601 1983; which references are entirely incorporated herein by reference.

Further, RSK2 inhibitors/antagonists can function at the genetic level. Such antagonists include agents which decrease, inhibit, block or abrogate RSK2 expression, production or activity. Such an agent can be an antisense nucleic acid or sequence specific peptide nucleic acid. In addition, such an antagonist may interfere with rsk2 promoter activity. Further, such an antagonist can be a RSK2 mutant such as a mutant that functions as a competitive inhibitor which can be introduced and expressed in the cell where RSK2 activity is to be reduced. The mutant can be a full length derivative of RSK2 or fragments or derivatives of RSK2 as described above, such that expression of the mutant in a cell, inhibits the endogenous RSK2 activity. Such antagonists can be introduced into a cell by transfection, for example calcium phosphate precipitation or lipofection; or by infection with a virus or pseudovirus containing the desired construct, or by electroproration or by gene gun. The nucleic acid of the present invention can also be introduced into cells wherein the nucleic acid comprises naked DNA. The nucleic acid can be in linear or circular form. Methods of introducing nucleic acid into a cell are well known in the art.

In another embodiment, RSK2 expression can be inhibited or reduced by introducing into a cell with a polynucleotide encoding rsk2 antisense nucleic acid. Methods of inhibiting expression using antisense inhibition of expression are well known in the art, see for example Iversen et al., “Anti-Cancer Drug Design”, Vol. 6 pp. 531-538 and “In Vivo Studies With Phosphorothioate Oligonucleotides: Pharmacokinetic Prologue”; Iverson P. L. published by McMillan Press. 1991, 6531-6538. For example, the antisense molecule can hybridize to the endogenous rsk2 mRNA and prevent translation of said mRNA, thereby inhibiting or reducing expression of said RSK protein. RSK expression can also be inhibited or reduced by transfecting the cell with a polynucleotide construct encoding a transcriptional inhibitor such that transcription of rsk2 is inhibited or reduced. Such a transcriptional inhibitor interacts, for example, specifically with rsk2 promoter sequences, resulting in decreased transcription of rsk2, decreased RSK2 protein expression and thus decreased RSK2 activity.

Nucleic acid as used herein refers to DNA, RNA or peptide nucleic acid (PNA). As used herein, the term “Peptide Nucleic Acid” or “PNA” includes compounds referred to as Peptide Nucleic Acids in U.S. Pat. Nos. 5,539,082, 5,527,675, 5,623,049 or 5,714,331 (herein incorporated by reference). Further modifications of PNA are well known in the art. Furthermore, like DNA, the backbone of the PNA can be modified, for example, to comprise phosphono-PNA.

In another embodiment, the present invention encompasses introducing into a cell a nucleotide expression construct, wherein said construct encodes a modified form of RSK2. A modified form of RSK2 can include a dominant negative RSK2. Such a molecule can competitively bind the RSK2 target molecule without activating said target molecule.

Several vectors for use in such constructs are well known in the art. Furthermore, mechanisms of delivery of said constructs to an individual are well known in the art. For example, recombinant expression vectors which include synthetic or cDNA-derived DNA fragments encoding modified RSK2 molecules comprising DNA encoding a modified RSK2 protein operably linked to suitable transcriptional or translational regulatory elements derived from mammalian, microbial, viral or insect genes. Such regulatory elements include a transcriptional promoter, an optional operator sequence to control transcription, a sequence encoding suitable mRNA ribosomal binding sites, and sequences which control the termination of transcription and translation, as described in detail below. The ability to replicate in a host, usually conferred by an origin of replication, and a selection gene to facilitate recognition of transformants may additionally be incorporated. Operably linked indicates that components are linked in such a manner that expression of the DNA encoding a fusion protein is controlled by the regulatory elements. Generally, operably linked means contiguous.

Mammalian expression vectors may comprise non-transcribed elements such as an origin of replication, a suitable promoter and enhancer linked to the gene to be expressed, and other 5′ or 3′ flanking nontranscribed sequences, and 5′ to 3′ nontranslated sequences, such as necessary ribosome binding sites, a poly-adenylation site, splice donor and acceptor sites, and transcriptional termination sequences.

The transcriptional and translational control sequences in expression vectors to be used in transforming vertebrate cells may be provided by viral sources. For example, commonly used promoters and enhancers are derived from Polyoma, Adenovirus 2, Simian Virus 40 (SV40), and human cytomegalovirus. DNA sequence derived from the SV40 viral genome, for example, SV40 origin, early and late promoter, enhancer, splice, and polyadenylation sites may be used to provide the other genetic elements required for expression of a heterologous DNA sequence. The early and late promoters are particularly useful because both are obtained easily from the virus as a fragment which also contains the SV40 viral origin or replication (Fiers et al., Nature 273:113, 1978. Smaller or larger SV40 fragments may also be used, provided the approximately 250 bp sequence extending from the Hind III site toward the BglII site located in the viral origin or replication is included. Exemplary vectors can be constructed as disclosed by Okayama and Berg (Mol Cell Biol 3:280, 1983.

Preferred eukaryotic vectors for expression of mammalian DNA include pIXY321 and pIXY344, both of which are yeast expression vectors derived from pBC102.K22 (ATCC 67,255) and yeast.

In another embodiment of the present invention it is desirable to increase the activity of RSK2, thereby resulting in weight gain and increased insulin sensitivity. RSK2 activity can be increased by introducing into a cell a nucleic acid construct expressing RSK2 or a biologically active fragment thereof. In this embodiment, the RSK2 protein, or biologically active fragment of RSK2, comprises a RSK2 protein or fragment with biological activity, comparable to the activity of endogenous RSK2, resulting in increased RSK2 activity.

The present invention further provides methods to identify molecules that modulate the RSK2 activity. Specifically encompassed by the present invention are methods to identify inhibitors/antagonists/agonists of RSK2 activity. Inhibitors of RSK2 activity can be identified and tested in in vitro assays and in ex vivo cell-based assays, as well as by in vivo assay as described herein.

Candidate inhibitors, such as peptides, small organic molecules or derivatives of downstream targets of RSK2, can be evaluated for their ability to specifically interact with RSK2 in standard binding or capture assays known in the art. Alternatively, candidate inhibitors can be selected for ability to specifically inhibit RSK2 kinase activity and/of inhibit the ability to RSK2 to act on its substrate.

In one embodiment, RSK2 can be immobilized to a suitable surface (such as wells of a plastic microtiter plate or on beads) and contacted under physiological conditions to a peptide library, organic molecule library or derivatives of RSK2 target molecules that have been labeled for subsequent detection. Molecules that bind RSK2 are selected and analyzed for their ability to inhibit RSK2 activity. In another embodiment, the peptide or small organic molecule library; the antibody or antibody fragments or the target molecule or target molecule derivatives can be immobilized on a solid support and contacted with RSK2.

Peptide libraries, such as an oriented peptide library (Z. Songyang et al. Cell 72:767, 1993; can be screened for peptides that interact with RSK2. Peptide libraries and other small organic molecule libraries can also be screened using other assays known in the art, such as proximity assays or Biospecific Interaction Analysis (BIA). Biospecific Interaction Analysis (BIA) in real time can be performed to evaluate candidate molecules for their ability to bind RSK2. Surface plasmon resonance (SPR), which is the basis for BIA measurements, is an optical phenomenon arising in metal films under conditions of total internal reflection. The phenomenon produces a sharp dip in the intensity of reflected light at a specific angle. The position of this resonance angle depends on several factors, including the refractive index of the medium close to the non-illuminated side of the metal film. Refractive index is directly related to the concentration of dissolved material in the medium. By keeping other factors constant, SPR is used to measure changes in the concentration of macromolecules in a surface layer of solution in contact with a dextran-coated gold film. Using the BIAcore™ instrument from Pharmacia Biosensor AB, the association and dissociation rate constants for a peptide or organic molecule binding to RSK2 can be measured. Polypeptides peptides, peptide mimics or small organic molecules exhibiting higher association constants (Kₐ) have the greatest potential for ability to interact with RSK2 and inhibit RSK2 activity.

The present invention includes cell lines suitable for use in the screening methods described herein. In one embodiment, the cell line is a mammalian cell line such as CHO cells, Ba/F3 cells, HepG2 cells or H35-hepatoma cells. In a another embodiment, the cell lines are derived from the rsk2 knockout mouse described herein and from a genetically identical mouse comprising wild type rsk2.

Candidate antagonists/agonists can be assessed for their ability to inhibit/enhance RSK2 activity comprising the steps of: culturing cells of interest under conditions suitable for maintenance and growth; contacting said cells with the candidate molecule or an organic molecule library comprising RSK2 inhibitors or transfecting the cells with a cDNA expressing the candidate molecule with a cDNA expression library comprising DNA encoding candidate RSK2 inhibitors; selecting the cells having increased RSK2 signaling activity and identifying the organic molecule or cDNA that had contacted the cells selected.

Candidate inhibitors/agonists can further be evaluated in animal models. In one embodiment, a rsk2 knockout mouse is treated with the inhibitor or agonist and evaluated for physiological effects such as weight gain, altered sensitivity to insulin or altered leptin levels. In another embodiement, mice that are wild type for RSK2 activity are treated with an inhibitor of RSK2 activity and evaluated for physiological effects such as weight loss, altered sensitivity to insulin or altered leptin levels.

Inhibitors identified as described by the present invention can be useful to treat obesity or prevent weight gain in a mammal.

The antagonists/agonists of the present invention can be formulated into compositions with an effective amount of the inhibitor/antagonist/agonist as the active ingredient. An effective amount of a RSK2 inhibitor/antagonist is an amount effective to partially or completely inhibit RSK2 activity. A useful alteration in activity can last from a few hours to many years after the administration of a single, multiple or continuous dose. An effective amount of a RSK2 antagonist/agonist is an amount affect to enhance RSK2 activity resulting in weight change. Methods to evaluate or increase RSK2 activity, such as monitoring, energy expenditure and weight for example gain/loss are well-known to those of skill in the art. It will be appreciated that the actual effective amounts of the inhibitor/antagonist/agonist in a specific case will vary according to the specific compound being utilized, the particular composition formulated, the mode of administration and the age, weight and condition of the mammal, for example. Dosages for a particular mammal can be determined by one of ordinary skill in the art using conventional considerations, (e.g. by means of an appropriate, conventional pharmacological protocol).

Such compositions can also comprise a pharmaceutically or physiologically acceptable carrier, and are referred to herein as physiological compositions. The compositions of the present invention can be administered intravenously, parenterally, orally, by transdermal patch, by inhalation or by suppository. The inhibitor/antagonist/agonist composition may be administered in a single dose or in more than one dose over a period of time to achieve a level of inhibitor/antagonist/agonist which is sufficient to confer the desired effect.

Suitable physiological carriers include, but are not limited to water, salt solutions, alcohols, polyethylene glycols, gelatin, carbohydrates such as lactose, amylose or starch, magnesium stearate, talc, silicic acid, viscous paraffin, fatty acid esters, hydroxymethylcellulose, polyvinyl pyrolidone, etc. The physiological preparations can be sterilized and if desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. They can also be combined where desired with other active agents, e.g., enzyme inhibitors, to reduce metabolic degradation.

For parenteral application, particularly suitable are injectable, sterile solutions, preferably oily or aqueous solutions, as well as suspensions, emulsions, or implants, including suppositories. Ampules are convenient unit dosages.

The inhibitors/antagonists/agonists of the present invention can be administered to an individual mammal in need of such treatment, in conjunction with an agent or agents that allow the inhibitor to pass through the blood brain barrier. The inhibitor/antagonist/agonist and the agent can be administered simultaneously or sequentially. Such agents are known in the art, such as those described in U.S. Pat. Nos. 5,112,596; 5,268,164; 5,686,416 and 5,506,206; the teachings of which are incorporated herein by reference in their entirety.

The following Examples are offered for the purpose of illustrating the present invention and are not to be construed to limit the scope of this invention.

### EXAMPLES

General Methods

RSK2 TISSUE DISTRIBUTION. The following tissues were rapidly dissected from a WT mouse and frozen in liquid N₂: heart, skeletal muscle, liver, fat, kidney, brain, intestine, spleen and lung. Tissues were homogenized and immunoblotted for RSK2 (see below).

IMMUNOBLOTTING. Muscle was polytron homogenized in ice-cold lysis buffer (20 mM Hepes, 2 mM EGTA, 50 mM β-glycerol phosphate, 1 mM DTT, 1 mM Na₃VO₄, 1% Triton X-100, 10% glycerol, 10 μg/mL aprotinin, and 1 mM phenylmethylsulfonyl fluoride). Homogenates were rotated end over end at 4° C. for 1 hour and then centrifuged at 14,000×g for 1 hour at 4° C. The suprernatants were collected and the protein concentrations measured using the Bradford method. Muscle proteins (100 μg) were separated by SDS-PAGE and transferred to nitrocellulose membranes. For phospho-MAP kinase, ERK2, and Akt-phospho Ser⁴⁷³ measurements and the membranes were blocked in TBS with 0.05% Tween-20 (TBS-T), 0.01% NaN₃ and 5% BSA for 2 hours at room temperature. Membranes were incubated with antibody against phospho-specific MAP (1:1000), ERK2 (0.2 μg/mL) or Akt-phospho Ser⁴⁷³ (2 μg/mL) in TBS-T with 2.5% BSA and 0.01% NaN₃ overnight at 4° C. For RSK2 immunoblots, membranes were blocked in TBS-5, 5% milk, 0.01% NaN₃ and incubated with antibody against RSK2 (3 μg/mL), in TBS-T, 0.01% NaN₃, 2.5% milk. Blots were washed with TBS-T and then incubated with donkey anti-rabbit IgG antibody conjugated to horseradish peroxidase (diluted 1:2000 in TBS-T, 5% milk) for 1 hour at room temperature. Blots were washed in TBS-T and antibody binding was detected using enhanced chemiluminescence.

IMMUNOPRECIPITATION. Muscle lysates were generated as above and 1 mg of protein was incubated with 10 μl of insulin receptor or IRS1 antiserum overnight at 4° C. Immune complexes were precipitated by adding 4 μl of protein A conjugated to agarose beads and incubating for 2 hours at 4° C. Immune complexes were washed twice in ice-cold wash buffer (50 mM Hepes, 100 mM NaF, 2 mM Na₃ Vo₄, 1% Triton X-100). Immune complexes were separated by SDS-PAGE and transferred to nitrocellulose membranes. The membranes were blocked in TBS-T, 0.01% NaN₃, and 5% BSA for 2 hours at room temperature. After blocking, the membranes from the ISR1 immunoprecipitation were cut at the 111 kD molecular weight marker and the top portion was immunoblotted for phosphotyrosine while the bottom portion was immunoblotted for p85. For insulin receptor and IRS1 tyrosine phosphorylation the membranes were incubated in anti-phosphotyrosine antibody diluted 1:1000 in TBS-T, 2.5% BSA, 0.01% NaN₃ overnight at 4° C. followed by 1 hour incubation in a rabbit anti-mouse IgG secondary antibody (5 μg/mL in TBS-T, 2.5% milk, 0.01% NaN₃ at room temperature. For IRS1-p85 association the bottom portion of the membranes from the IRS1 immunoprecipitation were incubated with antibody specific for the p85 subunit of PI-3 kinase diluted 1:500 in TBS-T, 2.5% BSA, 0.01% NaN₃ overnight at 4° C. Bound antibody was detected by incubating blots with ¹²⁵I-labeled protein A for 1 hour at room temperature. Blots were exposed on a phosphorimage screen.

### Example 1

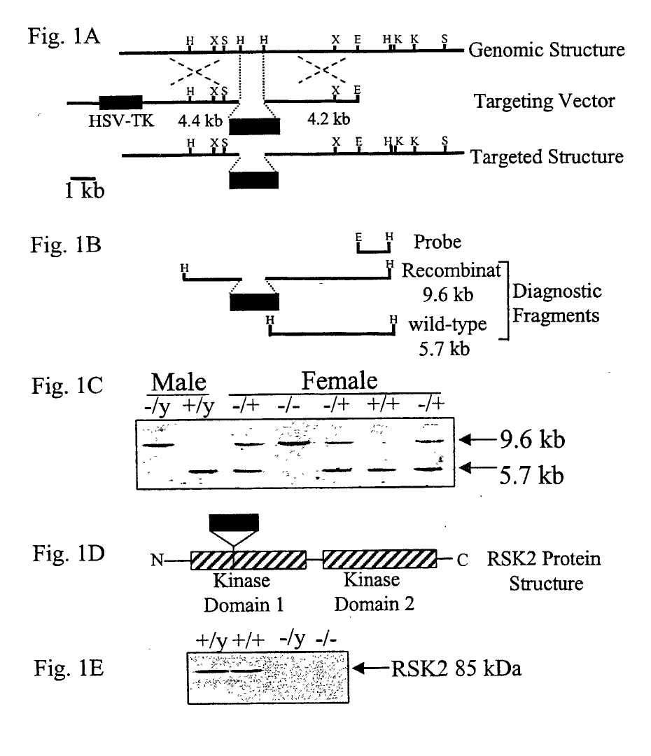

Mice lacking the RSK2 gene were generated (using homologous recombination). Briefly, a targeting vector was constructed, comprising a plasmid encoding the genomic version of rsk2 disrupted with a neomycin resistance gene and further comprising HSV-TK as shown in FIG. 1. A schematic diagram of the targeting plasmid is shown in FIG. 15. The plasmid contains the HSV-TK gene engineered for expression in ES cells. (Mansour et al. (1998) Nature 336:348-352). The TK gene is flanked by a duplication of a mutant Polyoma virus enhancer, PYF441, and has been inserted into the vector, pIC19R (Marsh et al. (1984) Gene 32:481-485) between the XhoI and the Hind III sites. The enhancer sequence is as follows:

5′

| | |
|---|---|
| 5′CTCGAGCAGT GTGGTTTTCA AGAGGAAGCA AAAAGCCTCT CCACCCAGGC | (SEQ ID No: 1) |
| CTGGAATGTT TCCACCCAAT GTCGAGCAGT GTGGTTTTGC AAGAGGAAGC | |
| AAAAAGCCTC TCCACCCAGG CCTGGAATGT TTCCACCCAA TGTCGAG3′. | |

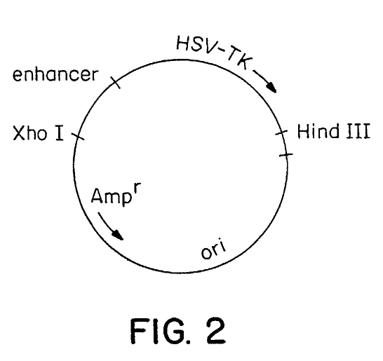

The 5′ end was an XhoI restriction site and the 3′ end was contiguous with the HSV-TK gene. The HSV-TK sequences were from nucleotides 92-1799 (McKnight (1990) Nucl. Acids Res. 8:5949-5964) followed at the 3′ end by a Hind III linker. The vector PIC 19R was essentially identical to the pUC vectors, with an alternative poly-linker. This plasmid was electroporated into ES cells using standard methods known in the art. Positive clones were identified by Southern blotting, wherein the wild type genomic copy of rsk2 revealed a 5.7 kb band and the disrupted rsk2 revealed a 9.6 kb band. Positive clones were used to generate a transgenic mouse line wherein both copies of rsk2 were replaced with disrupted rsk2, using methods well known in the art. The genotyping of the rsk2 knockout mice is shown in FIG. 1. As demonstrated in FIG. 1, rsk2 deficient mice do not produce RSK2 protein. As shown in FIG. 2, KO mice lack RSK2 production in muscle and white adipose tissue, while wild type littermates have RSK2 expression in muscle, and WAT. Furthermore, astrocytes and GT-7 cells also express RSK 2.

The knockout mice were studied for a number of physiological parameters, including their response to insulin. For insulin treatment to study ERK signaling, mice were injected intraperinoneally (i.p.) with 0.05 U/g body weight (B.W.) of procine insulin and control mice were injected with a corresponding volume of saline. Mice were killed by cervical dislocation 10 or 30 minutes after injection and gastrocnemius, soleus, and quadriceps muscles were dissected, pooled, and quick frozen in liquid N₂ [for studies of glycogen synthase, mice were anesthetized with an i.p. injection of pentabarbitol (90 mg/Kg body weight) followed by i.p. injection of 0.1 U/g B.W. procine insulin or a corresponding volume of saline. These mice were killed by cervical dislocation 15 minutes after the insulin injection. EDL muscles from both legs were pooled and used for glycogen and glycogen synthase measurements.] For exercise treatment mice were run on a treadmill for 60 minutes at 0.9 mph up to 8% grade while control mice were untreated. Hind limb muscles were rapidly dissected and frozen in liquid N₂. The gastrocnemius, soleus and quadriceps muscles from one leg were used for immunoblotting

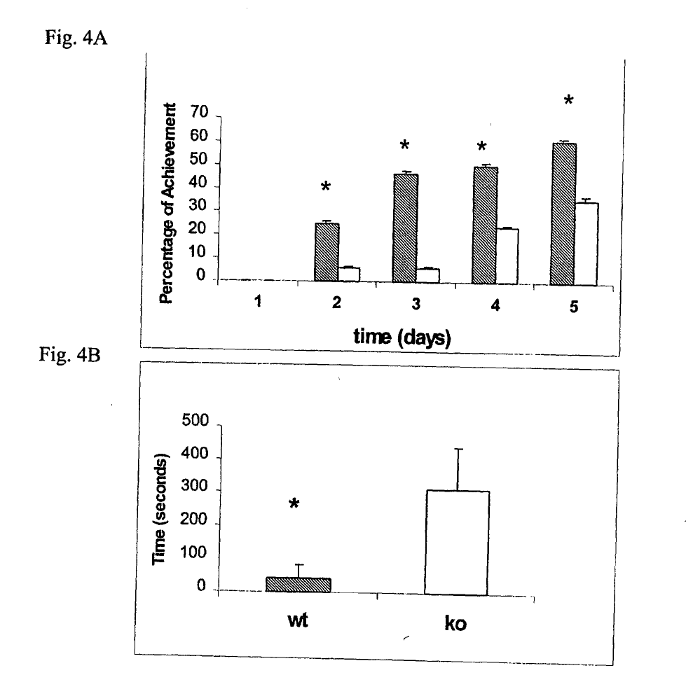

As shown in FIG. 4, KO mice showed less weight gain and smaller body size compared to their wild type littermates.

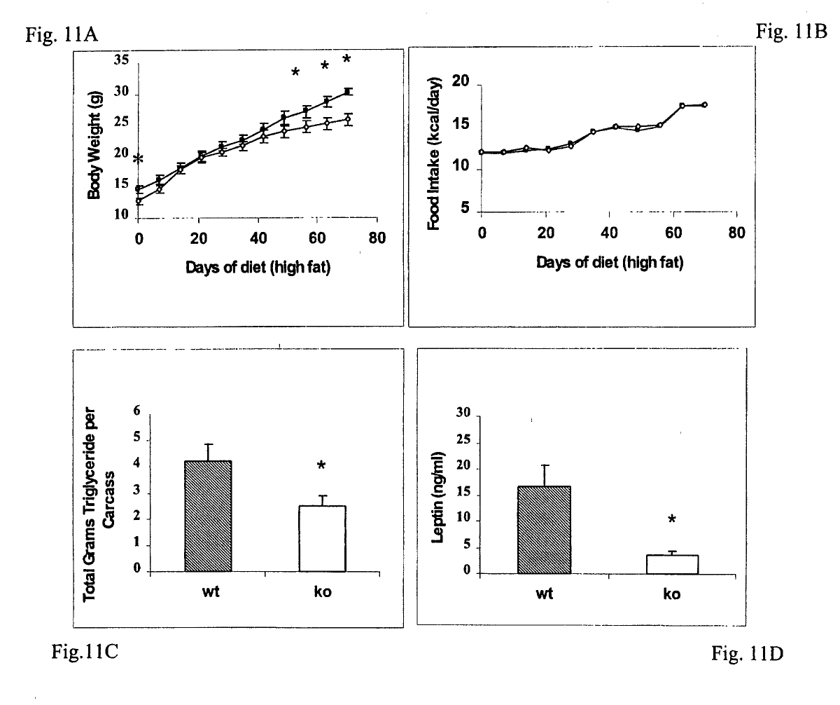

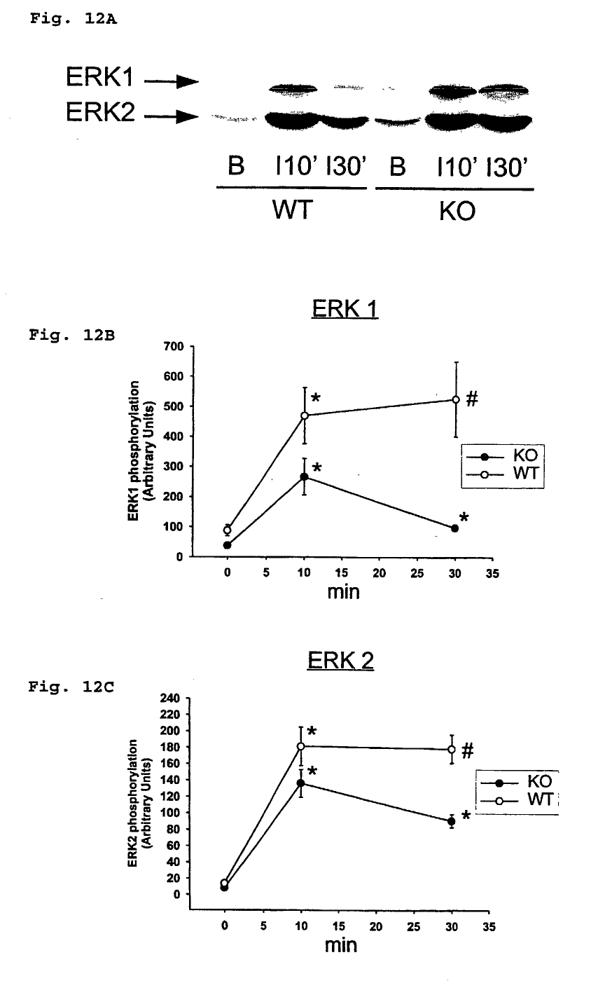

Hind limb skeletal muscles were obtained from mice 10, 15 or 30 minutes after insulin injection or immediately after strenuous treadmill exercise for 60 minutes. Mice were injected i.p. with 0.5 U/g B.W. of porcine insulin or with a corresponding volume of saline. Mice were killed by cervical dislocation (10 or 30 minutes after injection) and gastrocnemius, soleus and quadriceps muscles were dissected, pooled and quick frozen in liquid N₂. While insulin significantly increased MAP kinase phosphorylation in skeletal muscle from both WT and KO (FIG. 11), the increases were 2-fold greater in the KO animals (p<0.02). This occurred despite 33% lower MAP kinase expression in skeletal muscle of KO mice (p<0.0001).

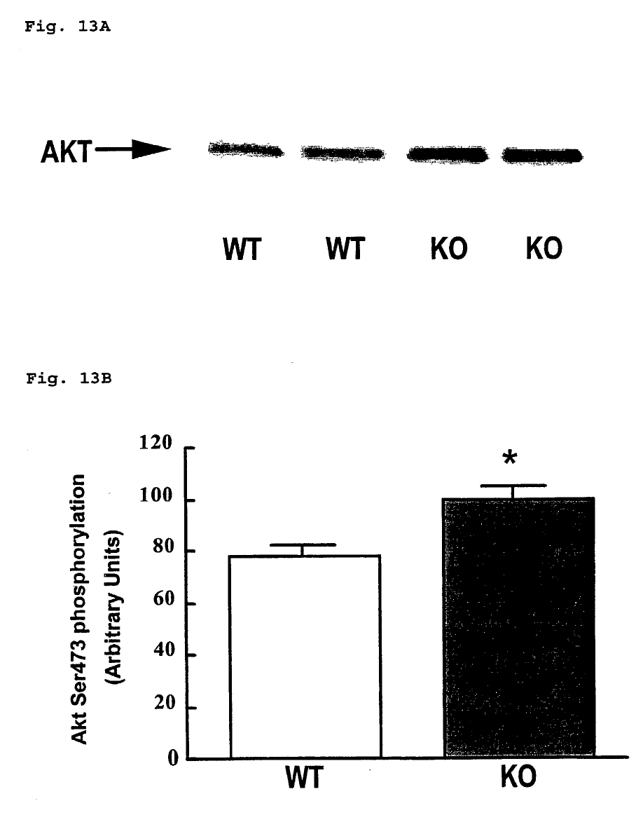

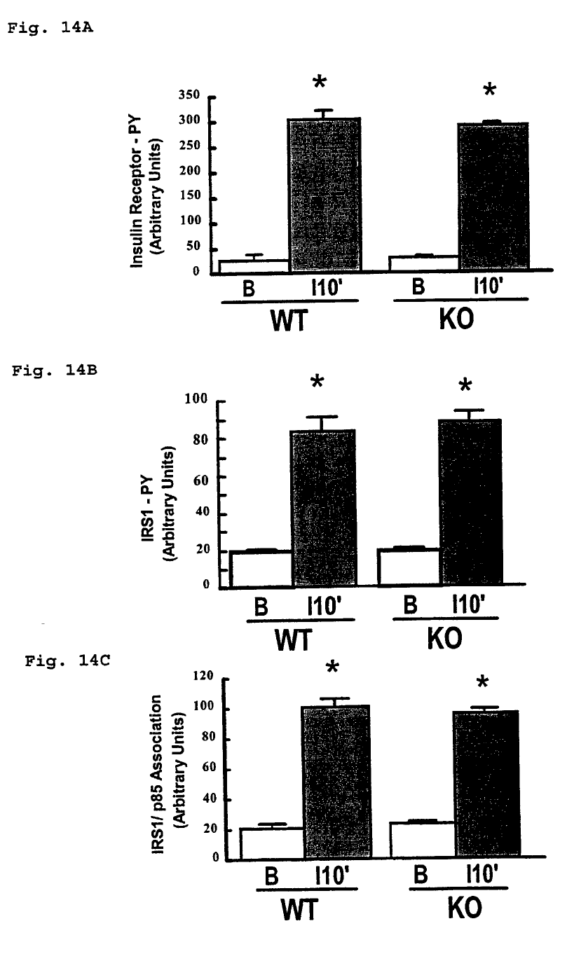

The enhanced insulin-stimulated increases in MAP kinase in KO mice were not associated with higher insulin receptor and IRS-1 tyrosine phosphorylation, nor IRS-1 binding to P1 3-kinase (FIG. 14). However, as shown in FIG. 13, insulin-stimulated Akt phosphorylation was significantly higher in the KO animals (p<0.02). It appears RSK2 is likely to have a role in feedback inhibition of the MAP kinase pathway and may also be involved in the regulation of Akt in skeletal muscle.

### Example 2

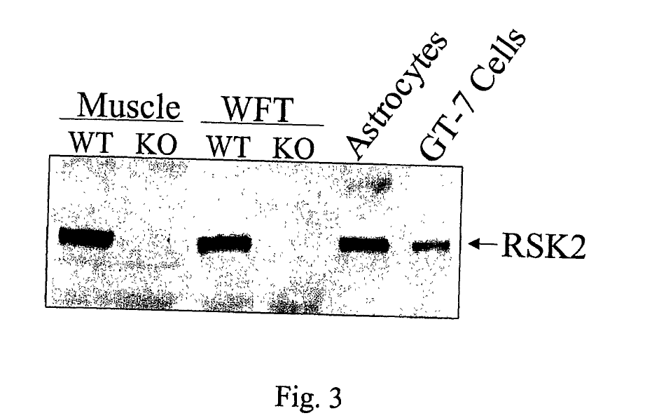

To further study the KO mice described above, other physiological parameters, such as neuroendocrine parameters were measured in these mice. Further, the learning capacity of the KO mice was tested. The learning capacity of the KO mice was lower as determined by the ability of the mice to learn to walk on a rotating stick (FIG. 3). In this experiment, 22 wild type and KO mice 20 weeks old were tested 3 times a day for 5 consecutive days for their ability to balance on a rotating stick. Each trial lasted 60 seconds. Wild type consistently demonstrated a greater rate of trial completion during the 5 day testing protocol. In contrast to the human manifestation of RSK2 deficiency (Coffin-Lowry syndrome) significant facial or digital abnormalities were not detected in rsk2-mice.

In a second test, 5 wild type and KO mice 10 weeks old were placed in a water-filled chamber and timed for their ability to locate a submerged platform on the opposite side. The time required for KO mice to locate the platform was nearly six-fold greater than the time required for wild type littermates, p<0.04, with data presented as mean +/− SE swim times from three separate experiments (FIG. 3B).

### Example 3

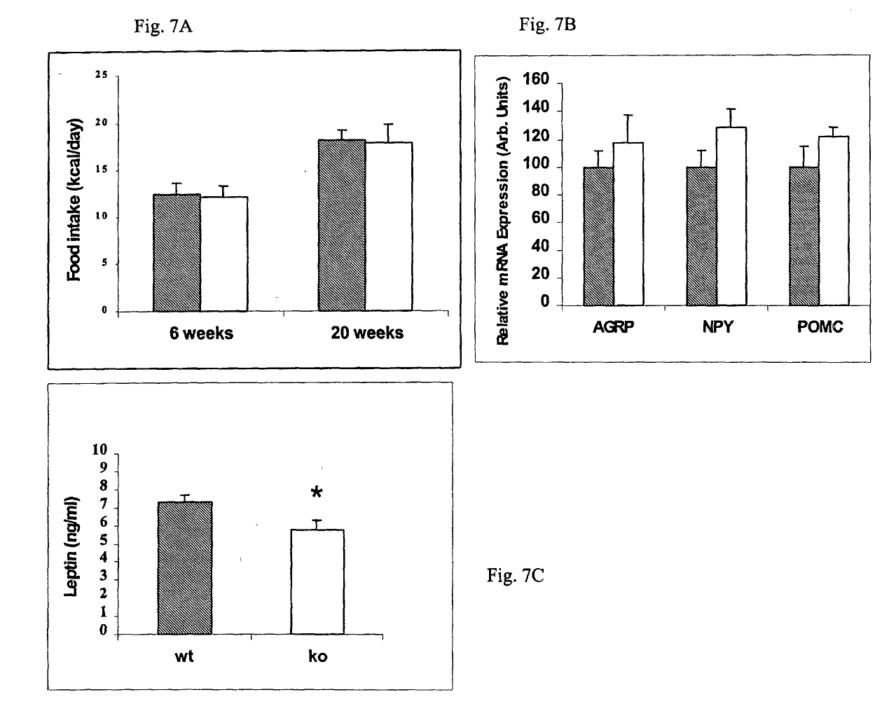

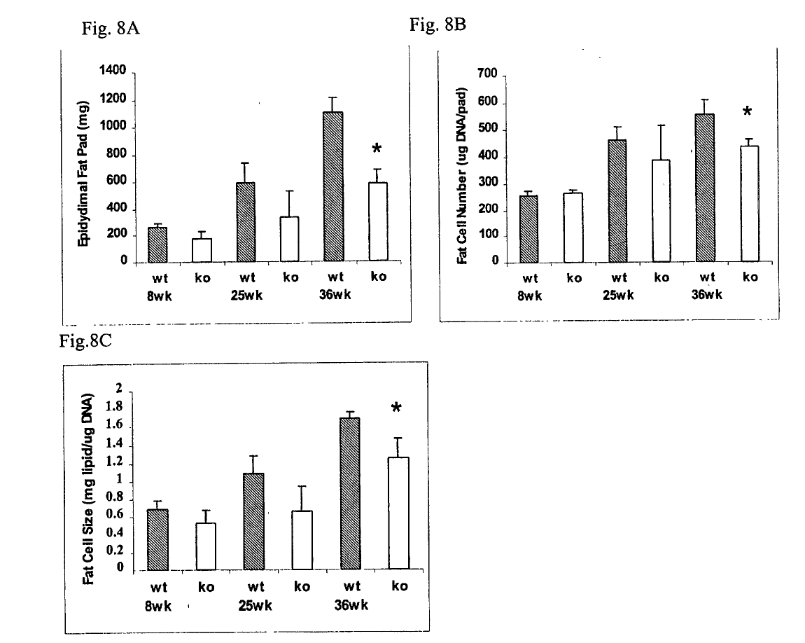

Fat pad weight (FIG. 7A), fat content in epididymal and renal tissue and total DNA and total lipid in epididymal fat was measured in WT and KO male mice at three different ages; 8, 25 and 36 weeks. The DNA content and fat pad weight measurements were used to estimate fat cell number (FIG. 7B) and the total lipid and DNA content were used to estimate fat cell size (FIG. 7C). As demonstrated in the Figures, at 8 weeks of age, there is no difference in any of the parameters between WT and KO mice. However, at 25 and 36 weeks of age, KO mice have much less fat than their wild type littermates and KO mice have a delay in the increase in fat pad mass. Furthermore, FIGS. 7A-C show that the reduced fat in KO mice is due both to fewer fat cells and smaller fat cell size.

### Example 4

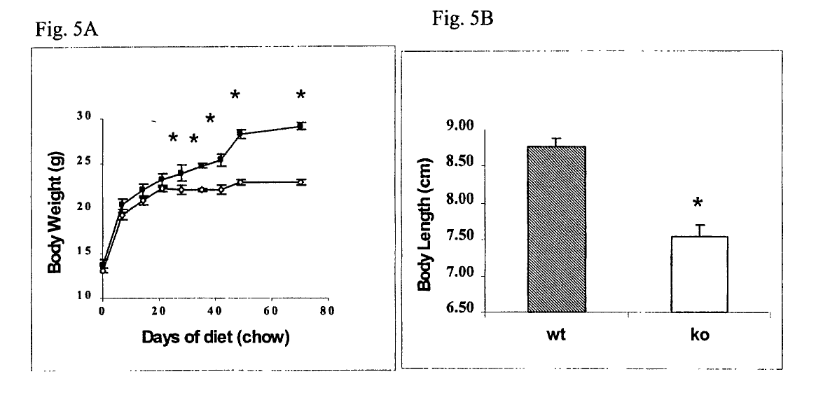

As shown in FIG. 5, KO mice showed similar body temperatures and UCP1, 2 and 3 mRNA expression levels in various tissues compared to wild type littermates. However, KO mice showed increased oxygen consumption compared to wild type littermates.

### Example 5

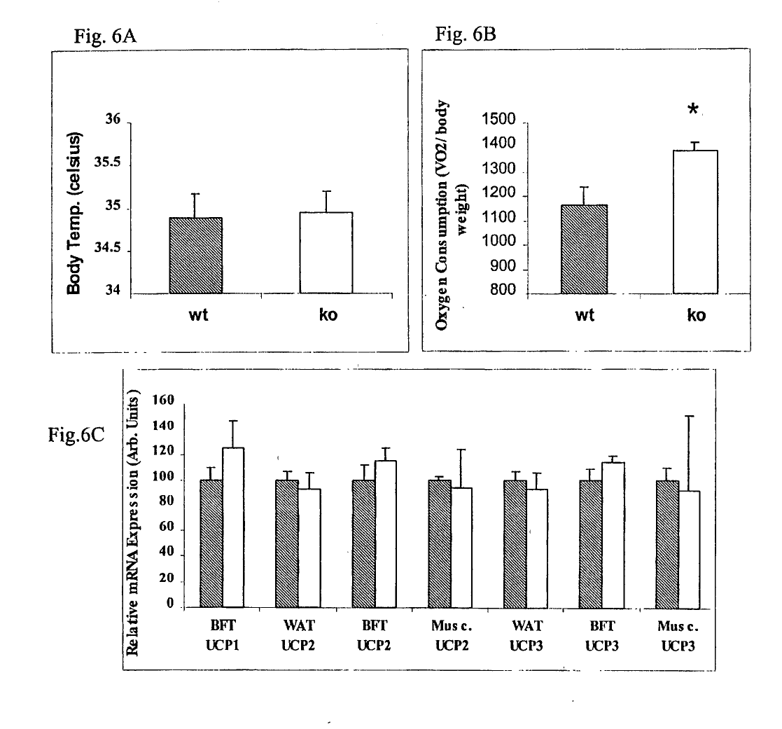

As shown in FIG. 6, KO mice and wild type littermates ate similar numbers of kilo calories per day. Total hypothalamic neuropeptide mRNA expression of neuropeptide Y (NPY), agouti-related peptide (AGRP) and proopiomelanocortin (POMC) were measured by quantitative RI-PCR on total RNA isolated from hypothalami. No differences between KO and wild type mice were detected. However, as also shown in FIG. 6, circulating serum leptin levels were significantly reduced in KO mice.

### Example 6

White adipose tissue (WAT) from wild type and KO mice were analyzed for activated Capase-3 by Western blotting. WAT from KO mice had detectable levels of activated Capase-3, a marker of apoptosis, while none was detected in WAT from wild type mice. These results suggest that KO mice have increased apoptosis in WAT. Therefore, one way to reduce adipose mass in a mammal is to induce apoptosis by decreasing RSK2 activity in adipose tissue.

### Example 7

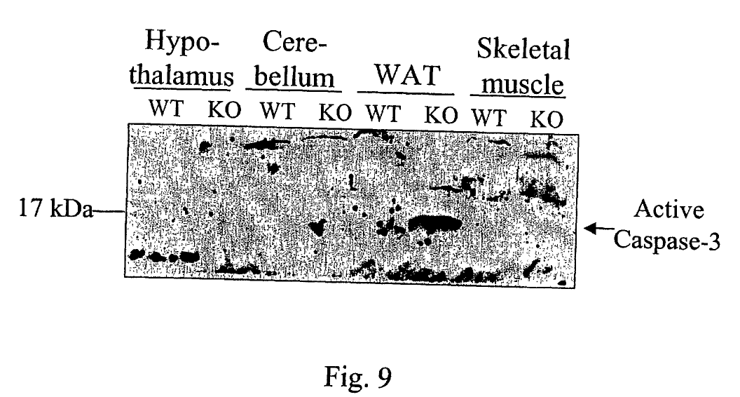

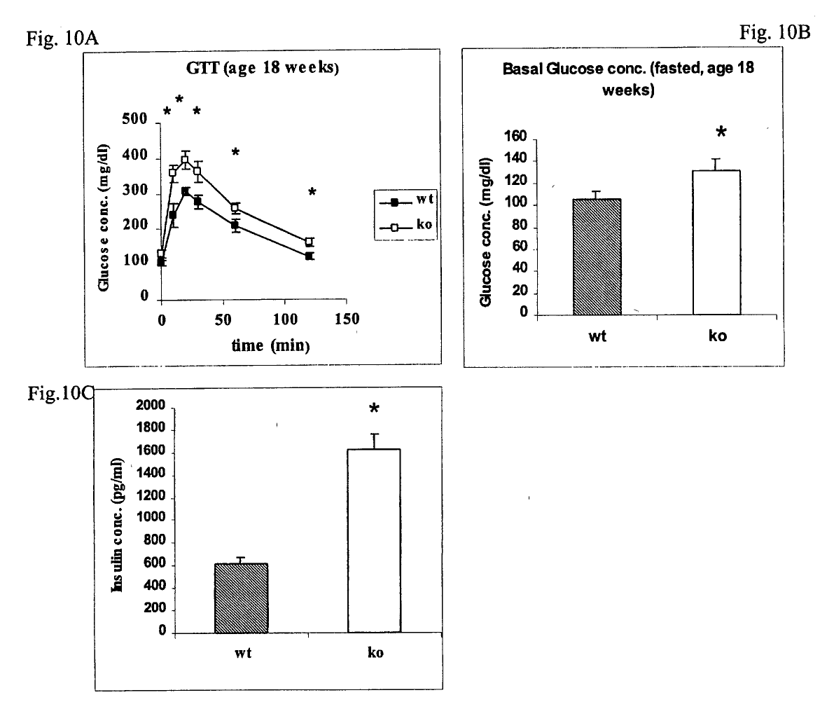

At 18 week of age on a chow diet, KO mice had an impaired glucose tolerance as measured by standard methods in the art (FIG. 9A). Fasting glucose and insulin levels were significantly elevated in rsk KO mice at this age, demonstrating insulin resistance in these mice (FIGS. 9B and C, respectively).

Wild type and KO mice were given a high-fat-containing (45% fat) diet (HFD) for 70 days. Body weight measurements revealed that KO mice had a reduced rate of weight gain as compared to wild type animals (FIG. 11A,*p<0.01). Food intake (kcal eaten per day) was not different between the groups (FIG. 11B). Total body fat was measured by carcass analysis after 70 days of HFD. A significant reduction in body fat was found in KO mice (FIG. 11C, *p<0.05). Leptin levels after 70 days of HFD were measure and KO mice were found to have a significant (>4 fold) reduction in serum leptin levels (FIG. 11D, *p<0.04)

### Example 8

To further study rsk2 KO mice, several physiological parameters were measured. Curculating corticosterone, T4, SDH, testosterone, leptin and IGF-1 levels were measured using standard assays. As shown in Table 1, leptin and IFG-1 levels were significantly reduced in KO mice using T-tests.

TABLE I

| | KO (n = 4 − 5) | WT (n = 8) |
|---|---|---|
| Leptin (ng/ml) | 5.7 +/− 0.6* | 7.25 +/− 0.43 |
| IGF-1 (ng/ml) | 11.0 +/− 9.3^{#} | 21.7 +/− 6.9 |
| Corticosterone (ng/ml) | 261.09 +/− 79.3 | 275.55 +/− 69.2 |
| T4 (μg/ml) | 2.94 +/− 0.57 | 3.14 +/− 0.78 |
| TSH (ng/dl) | 0.514 +/− 0.02 | 0.658 +/0.01 |
| Testosterone (ng/ml) | 336 +/− 65 | 411 +/− 89 |

*P < 0.01; #P < 0.02

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. Use of a RSK2 inhibitor for the manufacture of a medicament for:
a) treatment of obesity; or
b) reducing body weight; or
c) reducing body fat; or
d) reducing serum leptin levels; and
e) increasing oxygen consumption.

2. The method of claim 1 wherein the RSK2 inhibitor comprises;
a) polynucleotide construct comprises a polynucleotide that prevents transcription of rsk2 DNA; or
b) a polynucleotide encoding a rsk2 anti sense polynucleotide; or
c) a polynucleotide encoding a modified RSK2 polypeptide, wherein said modified RSK2 polypeptide is a competitive inhibitor of endogenous RSK2 activity; and
d) a RSK2 inhibitor, wherein the inhibitor interferes with the interaction of RSK2 with a RSK2 target protein.

3. A method of identifying inhibitors of RSK2 activity, comprising the steps of:
a) contacting cells having at least some RSK2 activity with an organic molecule library comprising candidate RSK2 inhibitors or transfecting said cells with a cDNA expression library comprising DNA encoding candidate RSK2 inhibitors;
b) selecting cells of a) having decreased RSK2 activity; and
c) identifying the organic molecule or DNA selected in step b).

4. A method of testing compounds in vivo for their ability to inhibit RSK2 activity comprising:
a) administering said compound to a mouse having at least some level of RSK2 activity;
b) measuring RSK2 activity in the mice treated in a);
c) selecting the compound that resulted in altered RSK2 activity.

5. A method of testing compounds in vivo for their ability to affect symptoms of Coffin-Lowry syndrome comprising:
a) administering said compound to a rsk2 knockout mouse;
b) measuring the effect on phenotypic parameters of Coffin-Lowry syndrome in mice treated in a);
c) selecting the compound that resulted in altered Coffin-Lowry phenotypic parameters.

6. The method of claim 5, wherein the phenotypic parameter is learning capacity.

7. A method of testing compounds in vivo for their ability to affect symptoms of lipodystrophy comprising:
a) administering said compound to a rsk2 knockout mouse;
b) measuring the effect on phenotypic parameters of lipodystrophy in mice treated in a);
c) selecting the compound that resulted in altered phenotypic parameters associated with lipodystrophy.

8. The method of claim 7, wherein the phenotypic parameters are selected from the group consisting of: adipose tissue levels, serum leptin levels, glucose sensitivity, body weight, insulin resistance and diet induced fat gain.

9. A compound identified by the method of claim 4.

10. A method of:
a) treating obesity;
b) reducing weight;
c) reducing body fat;
d) reducing serum leptin levels; or
e) increasing oxygen consumption in a mammal by inhibiting RSK2 activity in said mammal.

11. The method of claim 10, wherein the mammal is non-diabetic.
